(19) United States
(12) Patent Application Publication
(10) Pub. No.: US 2004/0005368 A1
(43) Pub. Date: Jan. 8, 2004

(54) Novel approach to weight loss comprising a modified protein composition that regulates blood sugar in conjunction with compositions that increase oxygen uptake and suppress appetite

(76) Inventors: Morris Mann, Glendale, AZ (US); Maria A. Mann, Glendale, AZ (US)

Correspondence Address: The Halvorson Law Firm, Ste. 1, 405 W. Southern Ave., Tempe, AZ, 85282, US

(21) Appl. No.: 10/187,668

(22) Filed: Jul. 1, 2002

(51) Int. Cl. A61K 35/78; A61K 33/24

(52) U.S. Cl. 424/725; 424/655

(57) ABSTRACT

Formulations and methods for enhancing lipolysis and the suppression of appetite are presented. Currently the preferred embodiment has these formulations as two separate compositions because of taste considerations (the combined taste, currently, is disagreeable). However, it is known that the two separate compositions can be combined into a single delivery systems, such as a drink, bar, gel or other nutritional delivery system known in the arts. The two separate compositions are: 1. compositions comprising substances that enhance oxygen uptake, and 2. a protein supplement composition comprising substances that regulate blood sugar. The overall purpose of this invention is to induce weight loss in as short of time as possible with the least amount of discomfort.

### FIELD OF THE INVENTION

The present invention is generally directed to mood stabilization, lipolysis, and appetite suppression, and compositions and methods relating thereto. These compositions include components that increase oxygen uptake and suppress appetite in conjunction with a modified food source that comprises protein compositions and compositions that regulate blood sugar.

### BACKGROUND OF THE INVENTION

Obesity continues to be a problem approaching pandemic proportions in this country. Unfortunately most weight loss approaches utilize substances that are inherently tachyphylactic. Historically substances that have induced weight loss have been amphetamines, their congeners, substituted tertiary amines and the like, or their precursors, mahuang cephedra and the like. Unfortunately, these substances promote anxiety when used at therapeutic levels and with increased use their efficacy proportionally diminishes. They also promote a secondary depression upon withdrawal due to neural transmitter depletion.

Thermogenic substances that promote lipolysis include a variety of different compounds and combinations that include, but are not limited to, ephedrine, xanthine compounds such as caffeine, theophylline, theobromine and alpha adrenergic stimulants. Yohimbine, coleus forskholii and the like or beta adrenergicstimulants such as clenbuterol.

However while these substances are thermogenic and lipolytic they have no sustained effect on appetite suppression. Interestingly, all the aforementioned, including amphetamines, affect oxygen transport by increasing bronchial diameter. In effect they are bronchodilators because they selectively affect relaxation of certain muscles in the bronchial tree. The addition, therefore, of a substance that selectively relaxes smooth muscle would further enhance this effect.

Surprisingly, alkaloids derived from coleus forskholii synergistically enhance bronchial dilation when combined with any of the aforementioned compounds, ephedra, xanthines and the like. This clearly enhances oxygen transport and therefore lipolysis. Yohimbine and its alkaloids can also effectively enhance bronchodilation in conjunction with ephedra or xanthine compounds.

Effective appetite suppression requires that the neural transmitters be in balance. This specifically includes the acetylcholine, serotonin, norepinephrine and dopamine pathways. In addition to increased appetites, imbalances in neural transmitters can also cause the symptoms of both depression and anxiety. It has been found that the vast majority of overeating occurs as a result of anxiety and/or depression. Thus, regulating the neural transmitter levels by appropriate supplementation will profoundly affect appetite and therefore sustain weight loss.

Anxiolytic preparations that are particularly useful include passion flower and magnesium containing compounds. Compounds that up-regulate acetylcholine by reversibly inhibiting acetylcholine breakdown are also effective in reducing anxiety. Physostigmine, pyridostigmine and Huperzine A are effective because they are acetylcholinesterase inhibitors and therefore they allow a build up of acetylcholine in the system. At the axial-dendritic junction, adding choline containing compounds such as dimethylaminoethanol (DMAE), phosphatidylcholine and/or choline bitartrate will obviously intensify the anxiety reduction effect because they provide the appropriate substrate precursors that will allow the body to manufacture acetylcholine.

Finally, inclusion of additional substances that have anti-depressive properties is useful to any composition that diminishes appetite. St. Johns Wort (Hypericum), buproprion hydrochloride and S-adenosyl methionine are useful examples of additional substances that affect and up regulate mood.

In order to supplement the bodies' ability to synthesize norepinephrine and dopamine, it is important to have the corresponding precursor amino acids, such as tyrosine and/or phenylalanine. The presence of these precursors will help prevent neural transmitter depletion and secondary depression so commonly seen after the cessation of ephedrine or xanthine compounds, said compounds cause global cerebrocortical stimulation and hence increase the utilization of the various neurotransmitters.

However, to truly and powerfully affect weight loss, the combination of an appropriate protein substrate in conjunction with the aforementioned substances that enhance oxygen uptake and prevent the global depression associated with the cerebrocortical stimulant effect would be most useful. In fact, the addition of a high protein, low fat, low carbohydrate supplement to the diet in conjunction with the aforementioned compositions, which will increase oxygen uptake, will dramatically and surprisingly affect weight loss. It will be clear that the combination of a substance that increases oxygen uptake in conjunction with a high protein supplement that regulates blood sugar is far more effective than either composition alone in inducing weight loss.

### SUMMARY OF THE INVENTION

The present invention provides an orally administered composition for enhancing lipolysis and inducing appetite suppression and methods provided thereto. Surprisingly it has been found that lipolysis and bronchial dilation with the attendant increase in oxygen transport are intimately related. Equally surprising it has been found that the use of said composition in conjunction with a high protein nutritional supplement that embodies an excess of methionine in conjunction with blood sugar regulating compounds such as inulin and chromium, dramatically enhances weight loss and thereby decreases fat in a much shorter time then would otherwise be the case with simple diet or the oxygen uptake enhancing composition alone. Of equal interest, it is noted that the combination of the oxygen uptake enhancing composition in conjunction with the high protein composition that regulates blood sugar tends to markedly decrease the sensation of hunger in those for that such obvious calorie restriction would other wise cause a significant degree of hardship. In fact it was determined by the use of the Hamilton Anxiety Scale that people on this particular approach were less anxious that they would be on either approach alone. As previously noted, anxiety and the attendant depression constitute the primary reasons for over eating in humans.

The novel features that are considered characteristic of the invention are set forth with particularity in the appended claims. The invention itself, however, both as to its structure and its operation together with the additional objects and advantages thereof will best be understood from the following description of the preferred embodiment of the present invention when read in conjunction with the accompanying figures. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DESCRIPTION OF THE DRAWINGS

FIG. 1 Average weight lost/week with Xanthine composition over 4 weeks.

FIG. 2 Average weight lost/week with Protein Supplement composition containing compositions that regulate blood sugar over 4 weeks.

FIG. 3 Average weight lost/week with Xanthine composition & protein supplement containing blood sugar regulating compositions over 4 weeks.

FIG. 4 Average weight lost/week with Ephedra & Xanthine & coleus forskholii over 4 weeks.

FIG. 5 Average weight lost/week with Ephedra and Xanthine and coleus forskholii and protein supplement composition over 4 weeks.

FIG. 6 Weight loss totals over 4 weeks according to the various compositions of FIGS. 1-5.

FIG. 7. Shows the results of the Hamilton Anxiety Test on subjects using the composition according to the present invention.

### DESCRIPTION OF THE PREFERRED EMBODIMENTS

The present invention is general directed toward compositions and methods for enhancing lipolysis and the suppression of appetite. Currently the preferred embodiment has these compositions as two separate compositions because of taste considerations (the combined taste, currently, is disagreeable). However, it is known that the two separate, compositions can be combined into a single delivery systems, such as a drink, bar, gel or other nutritional delivery system known in the arts. The two separate compositions are: 1. compositions comprising substances that enhance oxygen uptake, and 2. a protein supplement composition comprising substances that regulate blood sugar. The overall purpose of this invention is to induce weight loss in as short of time as possible with the least amount of discomfort. Although many specific details associated with certain aspects of the present invention are set forth below, those skilled in the art of pharmacology and especially neuro-pharmacology and nutrition will recognize that the present invention may have additional embodiments or that the invention may be practiced without several of the details disclosed herein.

Generally speaking, a diet is simply a reduction in caloric intake. The body responds to the lower caloric intake by in turn lowering metabolic rate in an attempt to conserve energy. Therefore, one can deduce that the body decreases its uptake of oxygen so as to decrease the amount of weight lost. At lower metabolic rates the body burns fewer calories than under normal circumstances, hence an individual gets disappointing weight loss results even though the caloric intake is lower. This is true of virtually all diets that last more than two to three weeks. If an individual wants to lose weight, reduce body fat, and improve muscle tone, more than diet is required. A total weight management and exercise program increases the body's metabolism and enables it to burn calories at a faster rate.

The components of the two separate compositions according to the present invention are more specifically described below.

The present invention provides a first separate composition that is an orally administered composition for enhancing oxygen uptake. There is also a second separate composition that provides is a nutritional protein supplement composition that regulates of blood sugar.

Oxygen Uptake Enhancing Composition

Exercise helps to increase the rate at which the body burns fat to thermogenicly/lipolytically produce heat. This is why exercise, in addition to moderate daily caloric intake, has always been an effective means for weight loss. Recently, the medical community has discovered that herbal thermogens help boost the body's ability to burn fat and curb an individuals appetite, thus making the addition of herbal thermogens a great adjunct to exercise and diet programs.

Several products on the market contain the herb ephedra, a versatile thermogenic/lipolytic herb. Ephedra contains ephedrine, a stimulant naturally found in the plant. This herb promotes weight loss because it has a thermogenic/lipolytic and fat-metabolizing effect. Ephedrine is thought to activate both alpha and beta adrenoceptors, which elevates metabolic rate, thereby increasing caloric expenditure and resulting in weight loss. Ephedrine also exhibits appetite suppressant properties by reducing one's desire for food. In other words, ephedrine simultaneously speeds up metabolism and reduces caloric intake by curbing the appetite.

It is important to remember that ephedra has been used for relieving asthma and allergies for many individuals. It is quite potent owing to the ephedrine and pseudoephedrine it naturally contains. Both compounds excite the sympathetic nervous system causing vasoconstriction of the nasal mucosa, dilation of the bronchioles, as well as cardiac stimulation. While these natural substances produce benefits similar to the body's adrenaline, they are not overly stimulating. This is why ephedra is such a useful herbal plant. It is extremely effective without being too strong in its actions when properly utilized.

The metabolic action of ephedra can be greatly enhanced when it is used in combination with a source of caffeine and other non-caffeine xanthines, such as when ephedra is combined with guarana and green tea alone. Caffeine, methylxanthines, and ephedrine work together to produce thermogenisis and to burn more fat than ephedrine alone. The greatest synergistic effect occurs when ephedra, guarana and green tea are combined with white willow bark (white willow bark is a source of salicin, a natural relative of aspirin). Some products designed to induce thermogenesis contain both ephedra and guarana plus occasionally white willow, but the fast acting synergistic combination of all four herbs yields the greatest results toward the goal of long lasting weight loss.

Thus, in the first separate composition contains caffeine in addition to at least one other member of the xanthine family that is not caffeine. The composition may contain caffeine and xanthine, caffeine and theophylline, caffeine and theobromine or caffeine, theophylline and xanthine, to name a few representative possibilities. In one embodiment, the composition contains caffeine and theophylline as the only active ingredients from the xanthines as defined above. The ratio of the weight of caffeine to the total weight of the other members of the xanthine family within the composition typically ranges from about 1:3 to 3:1, and preferably ranges from about 1:2 to 2:1.

The term “xanthine” as used herein refers to compounds incorporating the xanthine nucleus as shown below, wherein R¹, R², and R³ are independently selected from hydrogen and lower (C1-C4) alkyl.

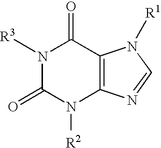

Exemplary xanthine compounds include xanthine, wherein R¹, R², and R³ are hydrogen; caffeine, also known as trimethylxanthine, where R¹, R², and R³ are each methyl; theophylline, which is also known as 1,3-dimethylxanthine, wherein R1 is hydrogen and R² and R³ are methyl; and theobromine, also known as 3,7-dimethyl xanthine, wherein R¹ and R² are methyl and R³ is hydrogen. As used herein, the term “first xanthine compound” means caffeine, and the term “second xanthine compound” refers to xanthine compounds as defined above, excluding caffeine. Pharmaceutically-acceptable salts, hydrates and solvates of xanthines are also included within the term “xanthines” as used herein. The salt may be an acid- or base- addition salt. Such salts may have at least one negatively charged ion such as chloride, bromide, sulfate, phosphate, C₁₋₁₅carboxylate, methanesulfonate and p-toluenesulfonate, where exemplary C₁₋₁₅carboxylate ions are acetate, glycolate, lactate, pyruvate, malonate, succinate, glutarate, fumarate, malate, tartarate, citrate, ascorbate, maleate, hydroxylmaleate, benzoate, hydroxybenzoate, phenylacetate, cinnamate, salicylate and 2-phenoxybenzoate. The salt may have at least one positively charged ion such as lithium, sodium, potassium, beryllium, magnesium, calcium and quaternary ammonium ions, where exemplary quaternary ammonium ions are tetraalkylammonium, and trialkylaralkyammonium ions. A solvate or hydrate of the salt may include ethylenediamine.

Xanthines are commercially available in pure form, and as such may be used in preparing compositions of the invention. For example, caffeine and theophylline are each available in 99% purity from Aldrich chemical Company (Milwaukee, Wis.), and may also be obtained from Sigma Chemical Company (St. Louis, Mo.).

Both caffeine and theophylline are known to have desirable effects on the mammalian body. Caffeine dilates coronary arteries and bronchioles in the lungs. In time, it also induces cerebral vasoconstriction and is a powerful neurostimulant. Theophylline increases bronchial dilation significantly, thereby enhancing the transportation of oxygen into cells and carbon dioxide out of the body, and is a low-grade cortical neurostimulant. Caffeine, however, is known to significantly enhance mental performance and to prolong a wakeful state. Both caffeine and theophylline are known to enhance physical performance.

The potentiating action of caffeine and salicin (found in white willow bark) on ephedrine's action has been studied in numerous weight loss studies in animals and humans. A report in the American Journal of Clinical Nutrition showed that when ephedrine was used alone with a group of animals, it resulted in losses of 14 percent in body weight and 42 percent in body fat. When it was used in combination with caffeine, however, there was a loss of 25 percent in body weight and 75 percent in body fat. In contrast, when caffeine was used alone there was no significant loss in body weight. The reason for the increased loss of body weight is an increased metabolic rate and fat cell breakdown promoted by ephedrine and potentiated by caffeine.

Research reported by Dr. Dulloo and others in Nutrition (5:7-9, 1989), has shown that when caffeine is combined with lose-dose aspirin and ephedrine, an ephedra/kolaut/white willow bark mixture has been shown to cause significant weight loss in overweight persons who consume a reduced-calorie diet.

It has been known for some time that small airway obstructions associated with clinical diseases such as asthma or emphysema have an inflammatory component. Surprisingly, the use of a known anti-inflammatory composition such as salicylic acid (naturally found in white willow bark) in conjunction with bronchodilators also substantially increases oxygen transport, and consequently increases calorie burning, of course many other anti-inflammatory agents could be used to similar results.

Acetylcholine is a neurotransmitter necessary for normal conduction of nerve impulses between nerve endings. In addition, the use of a reversible acetylcholinesterase inhibitor (a compound that inhibits the enzyme acetylcholinesterase, which breaks down acetylcholine and renders it ineffective at nerve junctions) further enhances acetylcholine levels and prevents attendant breakdown of this neurotransmitter level, in spite of chronic stimulant use. Huperzine A, a derivative of Chinese moss, is a particularly effective natural acetylcholinesterase inhibitor.

Research has also shown that the herb coleus forskhollii also relaxes smooth muscle, thereby inducing or increasing bronchiodilation, thereby increasing oxygen transport. This, in conjunction with the ability of caffeine to increase free fatty acid release, will clearly result in an increased tendency towards lipolysis (the breakdown of fat).

It appears that the regular use of ephedra, caffeine, methylxanthines, coleus forskhollii and salicin in is relatively safe for most people. Ephedrine, especially in its herbal form, also appears relatively innocuous for individuals not in a high risk group, such as individuals with high blood pressure, heart disease, diabetes, those taking antidepressants, or certain other prescription or over-the-counter medications.

In order to produce optimal weight loss benefits, herbal thermogenic/lipolytic products containing ephedra, guarana, green tea and white willow bark must be properly combined in synergistic formulations that include appropriate neurotransmitter precursors, as discussed below.

Although some authors have questioned the value of caffeine, Dr. Daniel Mowrey, in his book Fat Management! has proven caffeine to be effective in supporting a continued supply of neurotransmitters. Thus, the inclusion of caffeine acts not only to boost the stimulant effect for of other components, but to help preserve the balance of neurotransmitters necessary for total well being.

Caffeine is used in many cultures as a stimulant. Coffee is rapidly becoming our culture's “herbal” stimulant of choice. Studies show that most healthy people can safely consume up to 200 mg of caffeine and related methylxanthines per day without adverse reactions. Research shows that herbal thermogenic/lipolytic formulas are effective at levels well below this threshold.

It is also known that when used in combination methylxanthines, caffeine, and ephedrine, all of which will induce bronchodilation, has the effect of increasing weight loss that is demonstrably better than when the compounds are used alone.

One of the primary benefits of thermogenic/lipolytic formulas seems to be their ability to promote fat breakdown while sparing muscle tissue (since frequently low calorie diets also cause loss of muscle tissue). In one study reported by Astrup and coworkers in Metabolism (41:686-688, 1992), a combination of ephedrine (20 mg) and caffeine (200 mg), taken twice a day or a placebo for eight weeks in 16 obese women showed no significant difference in weight loss between groups. However, the ephedrine group lost on the average 4.5 kg more fat and 2.8 kg less muscle mass. While the total weight loss did not differ, the ephedrine group increased lipolysis while sparing muscle mass. Additionally, the ephedrine group had a higher level of energy expenditure than did the placebo group—a definite plus for dieting. The extra energy available for expenditure apparently resulted from the fat breakdown.

In addition to caffeine and a second (non-caffeine) xanthine, the first composition contains one or more cognitive cofactors. As used herein, a cognitive cofactor ameliorates diffuse chronic depolarization and subsequent cortical depression commonly associated with stimulants. Exemplary cognitive cofactors include, without limitation, biosynthetic precursors to neurotransmitters or neurosteroids, cerebral vasodilators, minerals, nootropic herbs, and essential amino acids.

The biosynthetic precursor of a neurotransmitter is a compound which, upon ingestion by a subject, is converted in vivo into a neurotransmitter, while a biosynthetic precursor of a neurosteroid is a compound, which upon ingestion by the subject, is converted in vivo into a neurosteroid. Biosynthetic precursors of both neurotransmitters and neurosteroids are well known in the art.

The following are exemplary cognitive cofactors according to the first composition: ginkgo biloba; niacin and derivatives containing the niacin nucleus; acetyl-L-carnitine; dimethylaminoethanol (DMAE); choline including esters and salts thereof, amino acids including salts and esters thereof, such as L-phenylalanine, glutamic acid, glycine, and aspartic acid; squalane; squalene; pregnenolone; dehydroepiandrosterone (DHEA); and dehydroeplandrosterone-3-sulphate. All of these biosynthetic precursors can be acquired from Sigma Chemical Company (St. Louis, Mo.).

A description of the above precursors follows:

Ginkgo biloba is a nootropic herb. It has been found to significantly increase cerebral circulation, enhance mental alertness, and increase the production of ATP in the brain. It also improves the ability of the brain to metabolize glucose. It is a powerful antioxidant and cerebral vasodilator.

Niacin and derivatives thereof include compounds that contain the niacin nucleus, which is shown below:

Niacin and derivatives thereof include, but are not limited to niacin, xanthinol nicotinate, methyl nicotinate, tocopheral nicotinate, and inositol hexanicotinate. Xanthinol nicotinate is a preferred niacin derivative and a preferred cognitive cofactor according to the invention. Xanthinol nicotinate is known to be a potent cerebral vasodilator of significant specificity, has been used for many years to lower serum cholesterol, and has been shown to dramatically enhance cerebral blood flow. On the basis of in vivo testing, it is also known that once inside brain cells, xanthinol nicotinate will increase glucose metabolism and correspondingly increase ATP. Xanthinol nicotinate does not generally cause flushing.

Acetyl-L-carnitine is related to choline compounds both clinically and chemically. Acetyl-L-carnitine protects the brain from the effects of aging. It has been definitively shown to decrease the buildup of lipofuscin pigments that are found in the brains of aged mammals. A buildup of these fatty deposits in nerve cells is associated with reduction of cognitive powers and a decrease in the rate of depolarization of nerve cells. Acetyl-L-carnitine increases brain levels of choline-acetyl-transferase and acetylcholine, a vital neurotransmitter.

Dimethylaminoethanol, or DMAE, is normally present in small amounts in mammalian brains. DMAE is known for its ability to elevate mood, enhance memory, increase intelligence, and increase the rate at which learning is accomplished. DMAE may take some time to have its effect noticed when taken alone. DMAE works by accelerating the brain's synthesis of the neurotransmitter acetylcholine. In the present composition, DMAE acts synergistically to dramatically enhance the effects of the xanthine stimulants.

Choline esters and salts as presented in compositions of the present invention are biosynthetic precursors to acetylcholine. A preferred choline salt is choline bitartrate, which is a phospholipid that is the immediate biosynthetic precursor of acetylcholine. Choline is known for its ability to improve memory by increasing the amount of acetylcholine in the brain. Choline bitartrate is a preferred form of choline because of its water solubility, which makes it more readily absorbable on the basis of oral administration.

Glutamic acid esters and salts, as used herein, includes pyroglutamate and arginine pyroglutamate. Pyroglutamate is a glutamic acid compound that is present in very large amouns in the human brain, cerbral spinal fluid, and blood. Pyroglutamate is known to have a number of remarkable cognitive enhancing effects. Studies have shown that pyroglutamate will effectively treat alcohol-induced memory deficits in humans. It has been shown that pyroglutamate can be very effectively transformed in the brain into the neurotransmitter glutamine. Arginine pyroglutamate has been found to not only enhance cognition, but is also an excellent grown hormone releasing factor because it is carried far more efficiently across the blood brain barrier than arginine alone. Other glutamic acid compounds are also efficacious as neurotransmitter precursors.

Aspartic acid and esters and salts thereof includes, without limitation, the sodium and potassium salts of aspartic acid. Potassium aspartate is a preferred aspartic acid salt, which may be used to enhance the intracellular ionic balance in the central nervous system that may otherwise be depleted by various stimulants.

Squalane and squalene are immediate biosynthetic precursors of all steroid molecules, including neurosteroids, and can be converted as needed to pregnenolone and/or other steroids. Pregnenolone is a neurosteroid that is known to enhance memory function. It has been conclusively shown to decrease GABA (gamma-amino-butyric acid) activity and thereby enhance wakefulness. Dehydroepiandrosterone and dehydroeplandrosterone-3-sulphate are related neurosteroids that are known to stabilize cell membranes. In particular, they are known to affect astrocytes and the splingomyelin sheath.

Specifically, the pathways for the neurotransmitters, acetylcholine, norepinephrine, and dopamine are affected. In order to ameliorate this problem, it is known that supplementation with neurotransmitter precursors can be effective in maintaining physiological levels of the neurotransmitters in the body.

The use of certain amino acids such as L-tyrosine and L-phenylalanine has been found to be particularly important since these are precursors to norepinephrine and dopamine. The use of various choline containing compounds such as choline citrate, and dimethlyaminoethanol (DMAE), etc. have proven effective in supporting the continued supply of neurotransmitters.

Huperzine A works by a unique mechanism that has been scientifically discovered and reported in many research journals. It acts as a potent acetylcholinesterase inhibitor. As stated earlier, acetylcholine is the neurotransmitter in the brain that is responsible for carrying electrical impulses from one nerve to another. Acetylcholine is produced in the end sections of nerve fibers and packaged into small vesicles where it is stored until released by the nerve ending. Once the nerve ending has secreted acetlycholine, it persists for a few seconds. In a normal brain, the enzyme acetylcholinesterase serves a housekeeping function by breaking down the acetylcholine into an acetate molecule (from the “acetyl” part) and choline. The choline (a member of the B-vitamin family) is then transmitted back into the nerve ending to be used again to make acetylcholine. Frequently when individuals take stimulant preparations, the precursors for producing acetylcholine are decreased, leading to a deficiency of acetylcholine available at the nerve junctions. Even with this deficiency, the acetylcholine is still released by the nerve endings. Huperzine A stops the acetylcholinesterase from breaking down acetylcholine, thus preventing acetylcholine deficiency and improving mental function.

Hypericum, the active ingredient in St. John's Wort, is known to be an effective anti-depressant and anti-anxiety agent (anxiolytic). Its effect is due to the inhibition of serotonin reuptake. Serotonin is another neurotransmitter whose levels affect mood, memory, anxiety and perceived energy levels. This, in conjunction with enhanced acetylocholine levels, surprisingly inhibits anxiety related to the use of any stimulant. Additionally, improvements in even sub-clinical depression and anxiety decrease the excessive appetite often seen with these conditions.

The amino acid L-tyrosine (normally present in dietary intake) and/or L-phenylalanine are also important to controlling how the brain functions. The brain can use L-tyrosine to synthesize the neurotransmitters norepinephrine and dopamine, both of which are critical to the feeling of alertness and stability. The addition of L-tyrosine to the preparation assists the brain to stabilize levels of norepinephrine and dopamine that would otherwise be depleted by stimulant use.

Ephedrine, caffeine, and theophylline all have central nervous system stimulant effects. As a result, it is well known that tachyphylaxis is a side effect of these stimulants and that sub-threshold depression may occur with sustained use or abrupt withdrawal. This is due to generalized neurotransmitter depletion. Specifically, the acetylcholine, norepinephrine, and dopamine pathways are affected. In order to ameliorate this problem, it was found that supplementation of neurotransmitter precursors was surprisingly effective. In addition, supplying an acetylcholinesterase inhibitor was particularly effective.

The use of certain amino acids such as L-phenylalanine and/or L-tyrosine was found to be particularly important since these are precursors to norepinephrine and dopamine. The use of various choline containing compounds including, but not limited to, phosphatidyl choline, choline citrate, dimethylaminoethanol, and the like, as proven effective. In addition, the use of a reversible acetylcholinesterase inhibitor further enhances acetylcholine levels and prevents attendant breakdown in spite of chronic stimulant use. Huperzine A, a derivative of Chinese moss, is a particularly good acetylcholinesterase inhibitor, although obviously other related pharmacological agents such as physostigmine or pyrodostigmine would also prove functional.

Anxiety and depression are known to result in excessive consumption of food beyond the body's nutritional requirements and dietary norms. Oftentimes, these conditions are sub-threshold; i.e., not clinically apparent. It is now apparent that overeating can be traced to deficiencies in certain neurotransmitters; i.e., norepinephrine and/or serotonin. Logically, therefore, substances that decrease and/or alleviate depression or anxiety will be useful in preventing excessive dietary consumption. This in conjunction with enhanced acetylcholine levels surprisingly inhibits appetite anxiety related to the use of any stimulant. Anxiety can be decreased with use of a variety of anxiolytic compounds. These include, but are not limited to, benzodiazepines, Kava alkaloids, passionflower, valerian, and/or chamomile extracts, and the like. Obviously, other antidepressants could be used such as buproprion hydrochloride, fluoxetine, and the like.

Several mineral compositions are useful supplements in the formulation according to the present invention. They include magnesium compounds such as magnesium phosphate or magnesium carbonate. It is known that magnesium and potassium contribute to the relaxation of smooth muscle.

A first example formulation of the oxygen uptake enhancing composition according to the present invention, with ranges of ingredients is noted below:

| Ingredient | mg/capsule | | Range % |
|---|---|---|---|
| Ephedra E @ 8% | 12 | mg | 0.1-40 |
| Green Tea Extract @ 90% | 50 | mg | 0.1-40 |
| Theophylline | | | |
| Guarana Extract @ 90% Caffeine | 40 | mg | 0.1-40 |
| Coleus Forskholii extract @ 10% | 2.5 | mg | 0.001-20 |
| L-Tyrosine | 65 | mg | 0.1-50 |
| Dimethylaminoethanol | 50 | mg | 0.1-75 |
| Choline Citrate | 50 | mg | 0.1-75 |
| Huperzine-A | 0.009 | mg | 0.000001-5 |
| St. John's Wort @ 0.3% hypericum | 25 | mg | 0.1-50 |
| Passionflower Extract A | 30 | mg | 0.1-50 |
| Potassium Chloride | 50 | mg | 0.1-20 |
| Magnesium Phosphate Dibasic | 100 | mg | 0.1-80 |
| Chromium Arginate | 0.1 | mg | 0.001-20 |
| White Willow Bark (Salicylic Acid) | 30 | mg | 0.01-75 |

| | |
|---|---|
| Excipients | as necessary |

The first four ingredients are thermogenic/lipolytic compositions that effect smooth muscle relaxation in bronchioles. The second four ingredients are neurotransmitter replacements that prevent acetylcholine breakdown. St. John's Wort and Passionflower Extract A are appetite suppressants (as their central effect), the potassium, magnesium, and chromium salts are mineral replacements, and the White Willow Bark acts as an anti-inflammatory agent.

If indeed, bronchodilation and its attendant increase in oxygen transport are the cause for lipolysis, then relative increases in FEV, will be directly related to weight loss. Surprisingly, this is indeed the case. Therefore, thermogenesis and resultant increases in metabolism are a direct consequence of increased oxygen delivery and consumption in either the resting or active state. To effectively demonstrate this principal, 10 individuals were given pulmonary function tests that measured FEV₁ and weight was measured and recorded before administration of the composition above. The 10 individuals then regularly took the composition above. One month later, FEV₁ and weight were re-measured. Interestingly, those with the largest positive increase in FEV₁ (indicating the greatest amount of bronchodilation) consistently lost the most weight.

In general, to achieve the beneficial results described above, a person in need thereof may be administered active ingredients of the first composition in an amount ranging from about 0.1 mg per kg of body weight per day to about 100 mg/kg/day. For the average person, a typical daily dosage is an amount ranging from 10 mg to 500 mg of caffeine in combination with a second xanthine compound (not caffeine) in an amount ranging from 1 mg to 1000 mg. The cognitive cofactor is present in a typical dosage in an amount ranging from 1 mg to 1000 mg. Preferred composition contains from 50 mg to 250 mg caffeine, from 10 to 500 mg of the second xanthine compound, and from 10 mg to 500 mg f the cognitive cofactor.

The composition formulated for oral administration should generally contain at lest about 4% of the active ingredients as identified above, but that amount may be varied up to 100% of the weight of the unit, if desired. The amount of the active ingredients present in orally-administered composition is such tat a suitable dosage will be obtained.

Preferred compositions and preparations according to the present invention are prepared so that an oral dosage unit form contains between 5.0-300 mg of the active ingredients as identified herein.

A second example formulation of the oxygen uptake enhancing composition according to the present invention, with ranges of ingredients is noted below:

| | Ingredient | mg/unit | Range (%) |
|---|---|---|---|
| 1. | Gingko A | 5.0 | |
| 2. | Theophylline | 25.0 | |
| 3. | Caffeine | 27.5 | |
| 4. | Green Tea | 84.0 | |
| 5. | L-pyroglutamate | 75.0 | |
| 6. | Xanthinol nicotinate | 38.0 | |
| 7. | N-Acetyl-L-carnitine | 7.5 | |
| 8. | Choline Bitarirate | 122.0 | |
| 9. | DMAE bitartrate | 60.0 | |
| 10. | Magnesium glycinate | 25.0 | |
| 11. | L-phenylalanine | 50.0 | |
| 12. | Chromium arginate | 200 | |

An efficacy trial was performed according to the following protocol: Three different groups of ten subjects were given a 240 calorie meal replacement. They were further given an additional composition, in capsule form, depending upon in which group they participated. The first group, Group A, was give a xanthine composition containing caffeine, theophylline and a cognitive co-factor (See the formulation below); the second group, Group B, was given the same amount of caffeine and theophylline noted in the xanthine composition given to Group A, but no cognitive cofactors; and the third group, Group C, was given a placebo. The subjects were weighed weekly for 6 weeks and asked to note how long they were able to go after ingesting the meal replacement and the capsules before feeling the need to eat again.

A third example formulation of the oxygen uptake enhancing composition according to the present invention, with ranges of ingredients is noted below:

| | Weight Loss Effect Formula | % Comp. | | Range % | |
|---|---|---|---|---|---|
| 1. | Caffeine | 220 | mg | 16.0% | 1-60 |
| 2. | Theophylline | 110 | mg | 8.0% | 1-60 |
| 3. | Gingko-A | 5 | mg | 0.36% | 0-50 |
| 4. | L-pyroglutamate | 100 | mg | 7.3% | 1-70 |
| 5. | Xanthinol nicotinate | 85 | mg | 6.2% | 1-70 |
| 6. | N-Acetyl-L-carnitine | 7.5 | mg | 0.55% | 0-50 |
| 7. | Choline Bitartrate | 122 | mg | 8.9% | 1-70 |
| 8. | DMAE | 200 | mg | 14.6% | 1-70 |
| 9. | Magnesium glycinate | 25 | mg | 1.8% | 0-50 |
| 10. | Potassium aspartate 21% | 50 | mg | 3.6% | 0-50 |
| 11. | Chromium arginate | 200 | mg | 14.6% | 0-5 |
| 12. | L-phenylalanine | 250 | mg | 18.2% | 1-70 |

The first composition provides, in one embodiment, a therapeutic composition for oral administration that includes caffeine, a second xanthine other than caffeine, and a cognitive cofactor as defined above. Thus, the invention provides compositions for oral administration that includes caffeine, a xanthine compound other than caffeine and ginkgo biloba; caffeine, a xanthine compound other than caffeine, and glutamic acid or ester or salt thereof; caffeine, a xanthine compound other than caffeine, and niacin or derivative thereof; caffeine, a xanthine compound other than caffeine and acetyl-L-carnitine; caffeine, a xanthine compound other than caffeine and dimethylaminoethanol; caffeine, a xanthine compound other than caffeine, and an amino acid or ester or salt thereof; caffeine, a xanthine compound other than caffeine, and L-phenylalanine; caffeine, a xanthine compound other than caffeine, and choline or a salt thereof; caffeine, a xanthine compound other than caffeine, and glycine or ester or salt thereof; caffeine, a xanthine compound other than caffeine, and aspartic acid or ester or salt thereof; caffeine, a xanthine compound other than caffeine, and squalane; caffeine, a xanthine compound other than caffeine, and squalene; caffeine, a xanthine compound other than caffeine, and pregnenolone; caffeine, a xanthine compound other than caffeine, and dehydroepiandrosterone; caffeine, a xanthine compound other than caffeine, and dehydroepiandrosterone-3-sulfate. In one embodiment, the xanthine compound other that caffeine is theophylline. In another embodiment, the niacin or derivative thereof is xanthinol nicotinate. In still another embodiment, the glutamic acid or ester or salt thereof is pyroglutamate. In yet a further embodiment of the invention, the afore-listed compositions contain only the mentioned compounds as active ingredients.

Blood Sugar Regulation Composition

The second separate composition is a nutritional protein supplement composition that includes long chain inulin and water soluble chromium compound for the regulation of blood sugar. It should be noted that water-soluble chromium containing compounds are far more effective in regulating blood sugar than standard non-water soluble chromium containing compounds, such as chromium picolinate. The preferred chromium containing compositions used in the present invention are chromium arginate and/or chromium chelidamate. The two chromium compounds noted above have the unique property of being water-soluble thereby making them considerable more bio-available than other chromium preparations.

It should be noted that if the diet is supplemented with a substance that enhances oxygen uptake, metabolic rate is not effectively decreased. Therefore weight loss proceeds at a more rapid and sustained pace than would otherwise be the case with either the oxygen containing composition alone or diet alone. A protein supplement composition with blood sugar regulating components is a novel addition to the first separate composition comprising either a xanthine composition or an ephedra based thermogenic compositions. It comprises a protein source, such as soy protein, whey protein, egg albumin protein, and the like. Preferably the protein supplement composition further comprises additional components such as inulin, L-methionine, MCT oil (a medium chain triglyceride oil, which is caprylic/caproic acid). Such components are administered to warm-blooded animals in need thereof in an amount sufficient to regulate blood sugar level and/or stabilize the mood of the animal. The preferred inulin used is a long chain derivative of chicory root with a molecular weight exceeding ten thousand. Compositions with these ingredients have been proven to be effective in regulating blood sugar in doses of anywhere between one and five grams per serving.

The second composition comprises inulin in addition to the source of protein. Inulin is a non-absorbable, non-nutritive carbohydrate that may be derived from natural sources, such as dahlia tubers, Jerusalem artichoke, or chicory root, or it may be synthesized. Prior to the isolation and purification of insulin, inulin was historically used by physicians and American Indians to regulate blood sugar levels in diabetic patients. To achieve a modicum of therapeutic regulation, a dosage of between 25 and 50 grams per day of inulin was required. These exceedingly large dosages have effectively precluded the usefulness of inulin administration for blood sugar regulation. Since that time, because of its unique non-absorbability it has been used to accurately determine renal clearance rates in normal and pathological states. Inulin is a complex carbohydrate consisting of beta-linked fructose subunits that may be represented by the formula (C₆H₁₂O₅)ₙ where n represents the number of fructose subunits in the carbohydrate and is indicative of the degree of polymerization. In the practice of the present invention, inulin with a degree of polymerization between 8 and 65 is preferred. In a more preferred embodiment, the inulin has a degree of polymerization between 15 and 45. Inulin is present in the compositions of this invention in an amount ranging from 10 to 99 percent by weight of the total composition, and preferably from 30 to 99 percent by weight of the total composition and the dosage range should be 200-800 mg.

Although not intending to be limited to the following theory, inulin, as a component of the composition of the present invention, serves to catalytically stimulate the 2,6, bisphosphate energy system. This, in turn, enhances and modifies glycogenolysis. Specifically, it is suspected that glycogen synthase a and glycogen phosphorylase b, which are not phosphorylated, in the presence of sufficient inulin behave as though they are phosphorylated. It is well known that the phosphorylation process is an active confirmation process that activates the catabolic enzymes that lead to glycogenolysis. Therefore, in the presence of inulin, glycogen stores will be utilized more efficiently. There is an interesting side effect that develops as a result of this process, to a greater extent than normal, carbohydrates are prevented from turning into storage fat.

The second compositions of the present invention, which contain inulin and a protein source are effective in regulating blood sugar levels at significantly lower dosages by virtue of the apparent synergistic affect of the other non-inulin composition components. These include certain metal complexes, as well as supplemental methionine. In the practice of the present invention, the dosage of inulin needed to effect blood sugar regulation ranges from about 50 micrograms (tg) to no more than about 10 grams of inulin per subject per day, and preferably from abut 1 gram to about 5 grams of inulin per subject per day.

As mentioned above, the inulin compositions of the present invention may include one or more metal complexes. The metal complex is present in the composition in an amount ranging from 0.01 to 20 percent by weight of the total composition, and preferably from 0.01 to 5 percent by weight of the total composition. These metal complexes, in conjunction with inulin, effect blood sugar regulation. Suitable metal complexes include metal complexes of chromium, manganese, and vanadium. As used herein, the term “complex” refers to any organic or inorganic ligated metal species.

While metal complexes alone generally have at least some capacity to effect blood sugar levels and improve glucose tolerance, the combination of inulin (in the amount disclosed above) and the metal complexes provide a composition that effects blood sugar level regulation significantly greater and at a much lower concentration that administration of either inulin or the individual metal complexes alone. Thus, the metal complexes are essential components of the compositions of the present invention.

For example, chromium is known to have some effect on glucose metabolism. These include, 1. Lowering blood levels of low density lipoproteins, 2. Raising high density lipoproteins, 3. Ability to modulate reactive hypoglycemia and 4. Modify exercise induced hypoglycemia. All of these effects are adequately documented. The effect of chromium on glucose metabolism was recognized as early as 1929 with the discovery that yeast extracts potentiated the effect of insulin and it is thought that a majority of the American public is chromium deficient. Seemingly, chronic chromium deficiency may be associated with an enhanced tendency towards atherosclerosis. In the presence of optimal amounts of biologically active chromium, much lower amounts of insulin are required. From an athletic point of view, this is a major advantage. Exercise can thus be conducted at higher intensity levels for longer periods of time before the induction of hypoglycemic fatigue. Several U.S. patents have disclosed the ability of chromium picolinate to influence blood sugar and insulin output (U.S. Pat. Nos. 5,164,384 and 4,315,927). In addition, it has been determined that the ability of mammalian tissue to absorb chromium decreases with age (see., e.g., Schroeder, The Trace Elements and Man, Devin-Adair, pub., Old Greenwich, Conn., 1977), and may explain, in part, maturity onset diabetes and its prevalence in humans after the age of 50.

Furthermore, some chromium complexes are known to have biological activity, including chromium trichloride, chromium acetate, chromium nicotinate (the active component of the metallovitamin, Glucose Tolerance Factor, isolated from yeast), chromium picolinate, chromium glycinate, chromium oxalate, chromium perchlorate, chromium salicylate, and chromium-4-oxo-pyridine-2, 6-dicarboxylate. Chromium is also a dietary requirement and chromium dietary requirements in humans range from about 50 to 200 μg per day.

Like chromium, manganese also improves glucose tolerance. Historically, glucose intolerance resulting from manganese deficiency was demonstrated in 1958. More recently, the importance of manganese in the diets of humans was demonstrated by Schroeder in 1966 (Schroeder et al., J. chronic Diseases 19:545-71, 1966). Although not formally listed as a required nutrient, manganese requirements in humans have been determined to be between 3 and 4 mg per day. Although manganese is poorly absorbed, the ability to absorb manganese does not decrease with age. The dietary dosage of manganese ranges from 2 to 100 mg per day.

Vanadium also effects blood sugar regulation and has recently been classified as an essential trance mineral. Vanadium complexes have been used in therapeutic applications including the treatment of diabetes. Vanadium is poorly absorbed and dietary intake ranges from about 2 to 15 mg per day. Because vanadium is poorly absorbed and its numerous complexes are extremely toxic, few vanadium complexes have been demonstrated to possess biological activity.

The chromium complexes of the present invention include organic and inorganic chromium complexes such as chromium acetate, chromium chloride, chromium potassium oxalate, and chromium potassium sulfate. In a preferred embodiment, the chromium complex is chromium picolinate. In a particularly preferred embodiment, the chromium complex is chromium-4-oxo-pyridine-2,6-dicarboxylate.

The manganese complexes of the present invention include manganese acetate, manganese chloride, manganese carbonate, potassium permanganate, dimanganese trisulphate, manganese gluconate, manganese glycinate and manganese citrate. In a preferred embodiment, the manganese complex is manganese gluconate or manganese glycinate.

Like the chromium complexes, the vanadium complexes include organic and inorganic vanadium complexes such as vanadium carbonyl, vanadium pentoxide, vanadium trisulfate, vanadyl dichloride, and vanadyl trichloride. Various organic vanadium complexes may also be used in the composition of the present invention. Examples of organic vanadium complexes include vanadyl glycinate, vanadyl gluconate, and vanadyl citrate. In a preferred embodiment, the vanadium complex is vanadyl sulfate (VSO₅).

The second compositions of the present invention optionally include medium chain triglycerides. As used therein, the term “medium chain triglyceride” (“MCT”) refers to a triester of glycerol containing medium length chain carboxylic acids. Medium length chain carboxylic acid chains are C₆ to C₁₂ carboxylic acids. The three medium chain carboxylic acids that are attached to the triglyceride backbone of the MCT may be, but need not be, the same. The medium chain carboxylic acids can be either saturated or unsaturated, but are preferably saturated. This unique fat in many respects behaves like a carbohydrate. It is absorbed directly into the splenic protal circulation where it is shuttled directly to the liver as free fatty acids bound to albumin. These medium-sized free fatty acids are preferentially oxidized to Acetyl-CoA, which can immediately enter appropriate bio-energetic pathways, especially the Krebs cycle. This is obviously advantageous in an energy depleted or active exercise state. Further, there is evidence that MCT's contributed to the stabilization of blood glucose during exercise. This supports the premise that excess energy as MCT is not stored with any degree of efficiency. In fact, a number of studies indicated that long-term feeding of MCT's at fairly high doses will paradoxically decrease plasma lipids and reduce fat deposition and body weight. Medium chain triglycerides are GRAS and have been used for more than 40 years in the feeding of premature infants both intravenously and orally.

Examples of medium chain carboxylic acids of this invention include C₆ (capric acid), and C₁₂ (lauric acid). As mentioned above, the MCT may bear one or more different carboxylic acid chains. In preferred embodiments, the MCTs comprise a mixture of from about 60% C₈ and about 40% C₁₀ to a mixture of abut 75% C₈ and abut 25% C₁₀. Odd numbered chains, such as C₇, C₉, and C₁₁ fatty acids, are less common, but are included within the scope of this invention. Further, the MCTs of the present invention may include minor amounts of short or long chain fatty acids. The medium chain tridgycerides are used in the present invention to reduce cravings for simple sugars that would otherwise increase insulin secretion. The medium chain triglyceride is optionally present in the composition in an amount ranging from 0 to 90 percent by weight of the total composition, and preferably from 0 to 67 percent by weight of the total composition.

In addition to the heating substances and protein, inulin, mineral complexes and MCT oil, the second composition may further include an amino acid selected from L-methionine, D-phenylalanine, glycine, and mixtures thereof. Preferably, the amino acid is L-methionine, a primary amine, and formed in high concentration in legumes. L-methionine is believed to selectively affect the appetite control center in the septal region of the hippocampus, resulting in a perception by the brain of significant food intake, and thereby producing a sensation of satiety. This presumably is due to the fact that methionine is the scarcest of all of the essential amino acids in food. D-phenylalanine is known to increase endorphin levels in the body and, since endorphins are released after a large meal, it is believed to contribute to a feeling of satiety. Glycine stimulates the release of glucagon, which raises blood glucose levels that have fallen too low. This aids in the prevention of overeating by those with hypoglycemia (low blood sugar). Thus, the presence of one or more of these amino acids in the composition imparts further advantages relating to appetite suppression. Preferably, the amino acid is present in the appetite suppressant composition in an amount ranging from 5 mg to 2000 mg.

A first example formulation of the Protein Composition and Blood Sugar Regulating Composition according to the present invention, with ranges of ingredients is noted below:

| | Ingredient | Wt. % | Range (%) |
|---|---|---|---|
| 1. | Soy Protein | 89 | 10-99 |
| 2. | Inulin | 4 | 0.01-20 |
| 3. | L-Methionine | 0.2 | 0.01-20 |
| 4. | MCT oil | 3.0 | 0-10 |
| | (medium chain Triglyceride | | |
| 5. | Vanilla Flavoring | 0.3 | 0-10 |
| 6. | Sucralose | 0.2 | 0-10 |
| 7. | Carboxymethyl Cellulose | 0.8 | 0-20 |
| 8. | Carrageenan | 0.4 | 0-20 |
| 9. | Magesium Phosphate | 1.198 | 0-30 |
| 10. | Chromium Arginate | 0.001 | 0-5 |
| 11. | Chromium Chelidamate | 0.001 | 0-5 |
| 12. | Glycine | 0.2 | 0.01-20 |
| 13. | Vanadyl Sulfate | 0.2 | 0.001-10 |
| 14. | Manganese gluconate | 0.2 | 0.001-10 |

In addition to the above-identified ingredients, the composition may contain optional ingredients. On optional ingredient is a stimulant, which is not one of the above-mentioned ingredients. In general, materials known to have a stimulatory effect are well known in the art, and any of these materials may be present in the composition of the invention. An exemplary stimulant is phenethylamine.

In another aspect of the present invention, a method for regulating blood sugar levels is disclosed. The method provides for the systemic administration of the compositions of the present invention in a quantity sufficient to regulate blood sugar levels in warm-blooded animals. In one embodiment, the inventive formulation of the present invention are administered to a warm-blooded animal in an oral form. When formulated as capsules, the inulin composition is preferably administered one to three times a day. While the oral dosage may contain from 100 mg to 6000 mg (e.g., total weight of all active ingredients), a single tablet or capsule containing more than about 1000 mg may be too large to easily swallow. Thus, the inventive formulation may be administered in either multiple capsule, multiple tablet form, powders, or a ready to drink beverage. In addition, The total weight of all active ingredients will depend on the form of ingredients used.

### Test Results

During the course of the study reported herein, all individuals had a morning and afternoon meals consisting of the high protein blood sugar regulating composition and an orange or an apple. In the evening they were instructed to eat a standard meal but to limit their intake of refined carbohydrates such as bread and rice. Upon arising and at 2:00 p.m. all participants took two capsules containing either the ephedra based or non-ephedra based anorectic broncho-dilator as noted by the formulas given above. In total, there were five different formulations administered to the individuals: 1. A composition containing a xanthine; 2. A composition containing the protein supplement and blood sugar regulator; 3. Both a xanthine containing composition and a protein supplement with blood sugar regulator; 4. A composition containing Ephedra, xanthine, and coleus forskholii; and 5. A composition containing Ephedra, xanthine, and coleus forskholii and the protein supplement with blood sugar regulator.

For the purpose of all experiments reported herein, individuals were given servings of two ounces of the protein composition in water, two times per day. Obviously, one skilled in the art could generate compositions that would be in solid forms such as bar or gel form that would be equally effective. This does not alter the spirit or the effect of the invention.

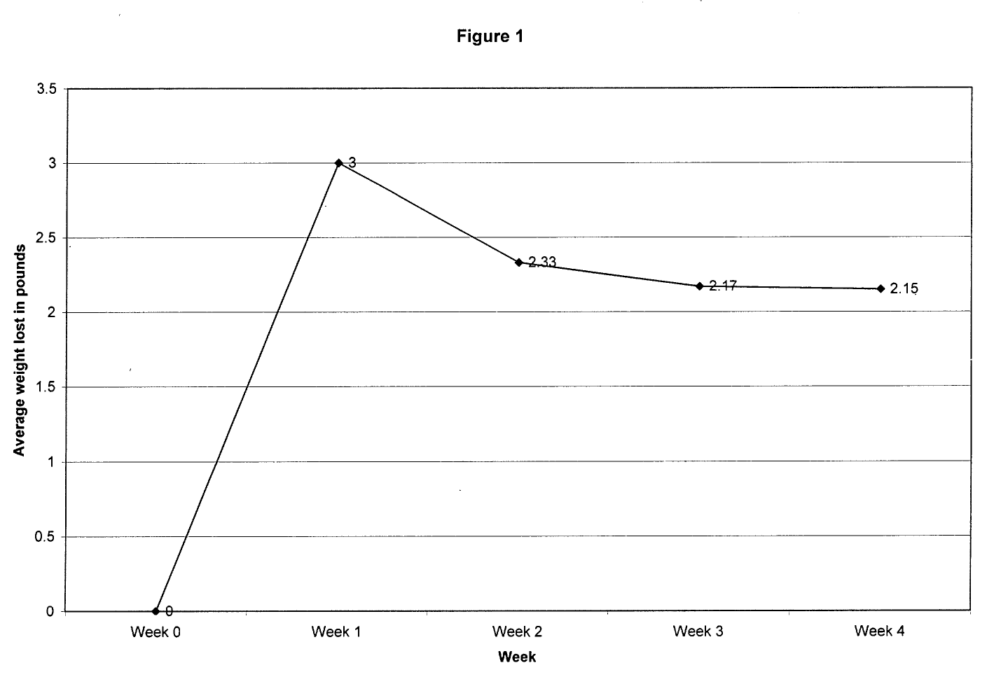

FIG. 1 shows the average weight lost (in pound) using a xanthine containing composition. This composition showed an initial 3 pound weight loss in the first week, that tapered down at about 2.15 pounds lost at the fourth week.

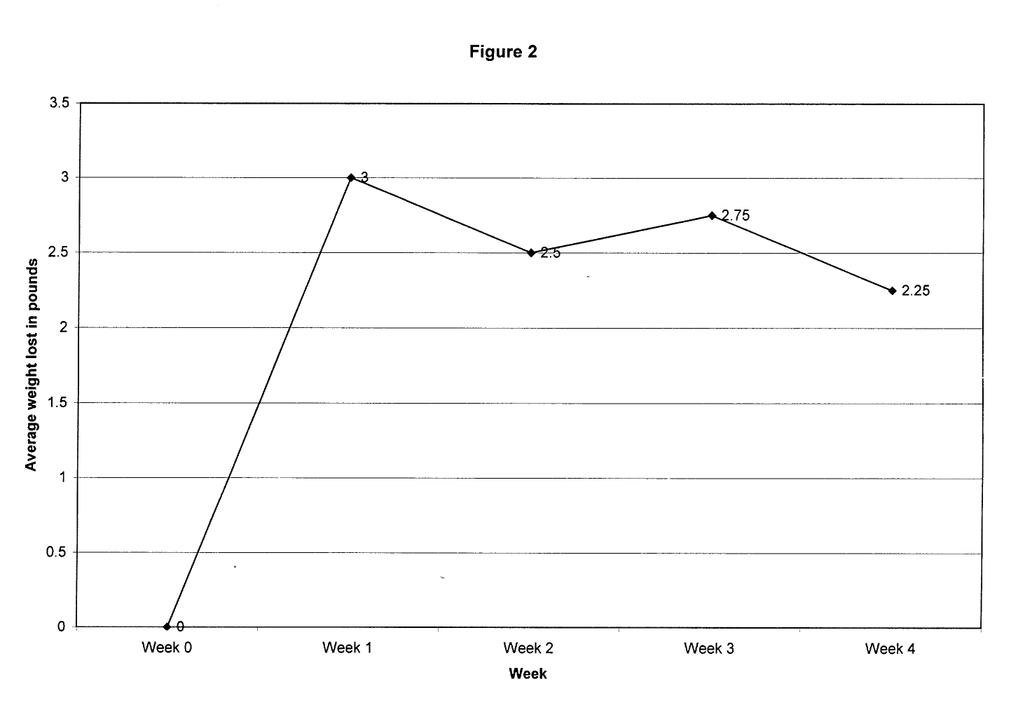

FIG. 2 shows the average weight lost (in pounds) using a protein supplement composition containing a blood sugar regulator. This formulation also showed an initial weight loss of 3 pounds in the first week, with the average tapering down to about 2.25 pounds by the fourth week.

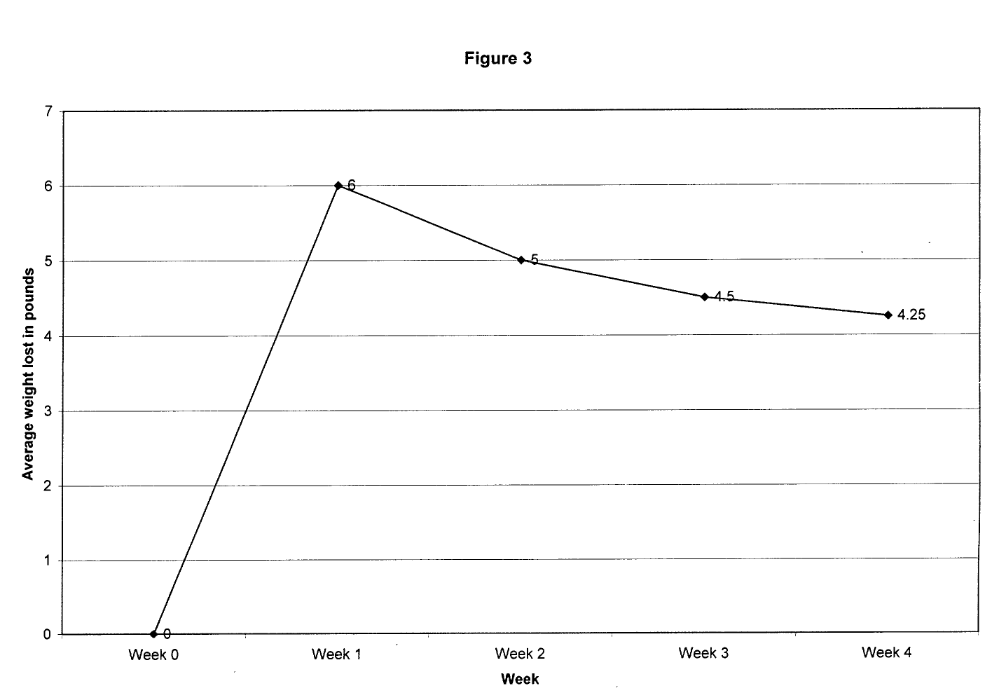

FIG. 3 shows the average weight lost (in pounds) using a xanthine containing composition and a protein supplement composition containing a blood sugar regulator. As can be seen from the figure, the initial weight lost in the first week was double that of the separate compositions individually, at 6 pounds. Further, this weight loss tapered down to about 4.25 pounds, still greater than that of the initial weight loss of the separate compositions and approximately twice that at the fourth week. This surprising and unexpected result supports the utility of the present invention.

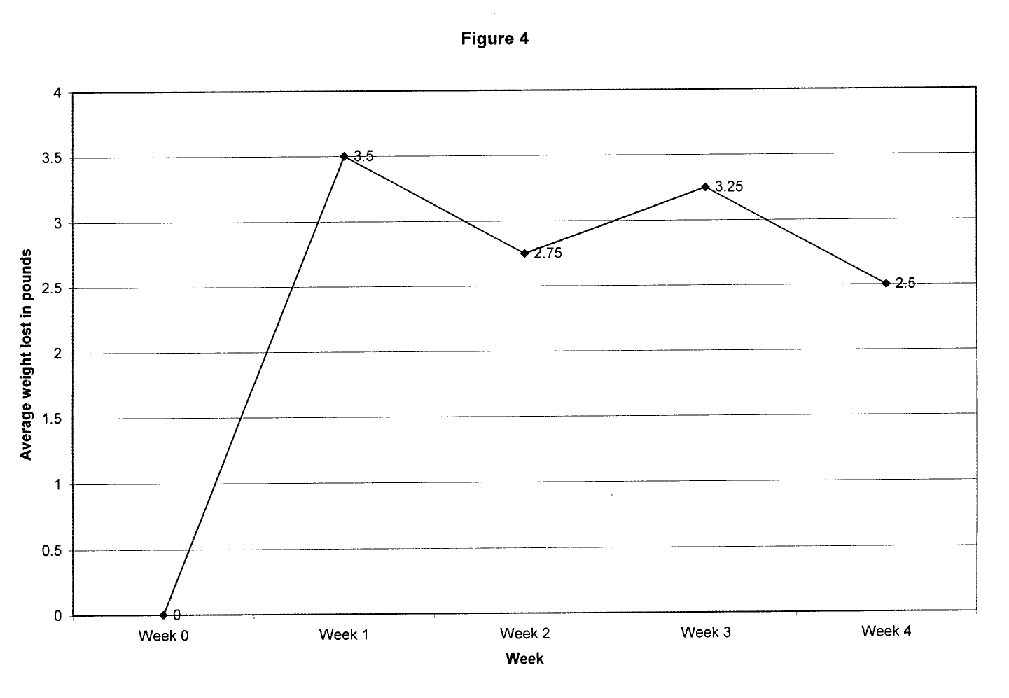

FIG. 4 shows the average weight lost (in pounds) using a composition containing Ephedra, Xanthine, and coleus forskholii. This composition performed better than the xanthine containing composition and protein supplement compositions, but not as well as the combination of the two. This formulation showed an initial weight loss of 3.5 pounds in the first week, which tapered off to about 2.5 pounds in the fourth week.

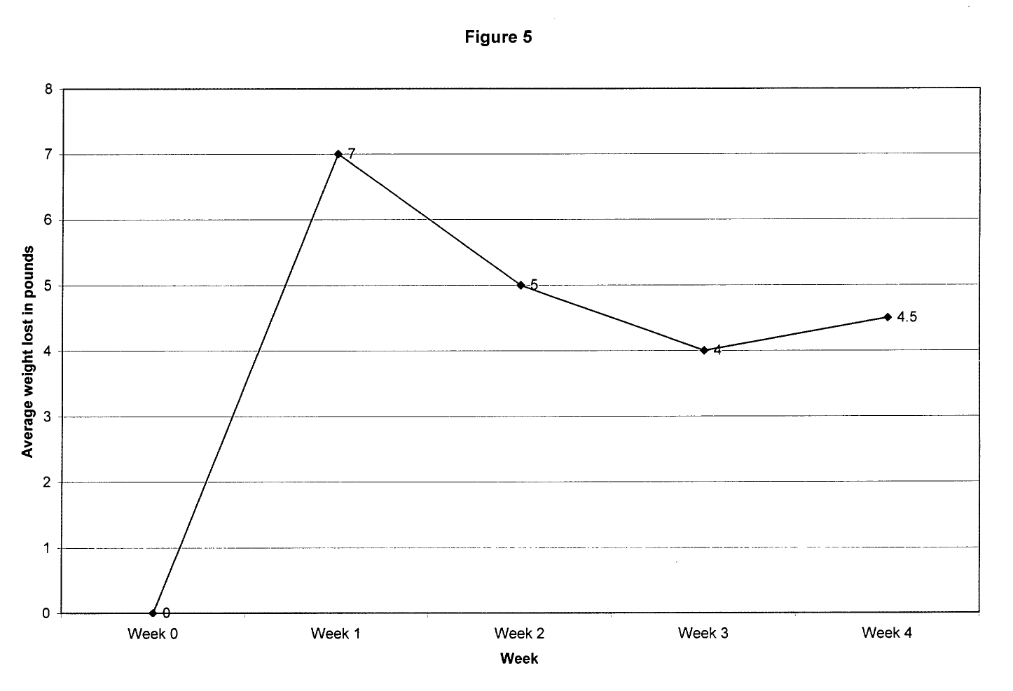

FIG. 5 shows the average weight lost (in pounds) using a composition containing ephedra, xanthine, and coleus forskholli and a protein supplement composition with a blood sugar regulator. This formulation showed the best results. There was a 7 pound initial weight loss that tapered of to about 4.5 pounds in the fourth week.

These figures clearly illustrate that the combination of a xanthine containing composition and a protein supplement composition containing a blood sugar regulator show surprising and unexpectedly superior results in weight loss experienced by the using individuals. Further, these figure clearly show that while coleus forskholii clearly improves the performance of the xanthine containing composition, it affects a greater improvement on the ephedra-xanthine formulations.

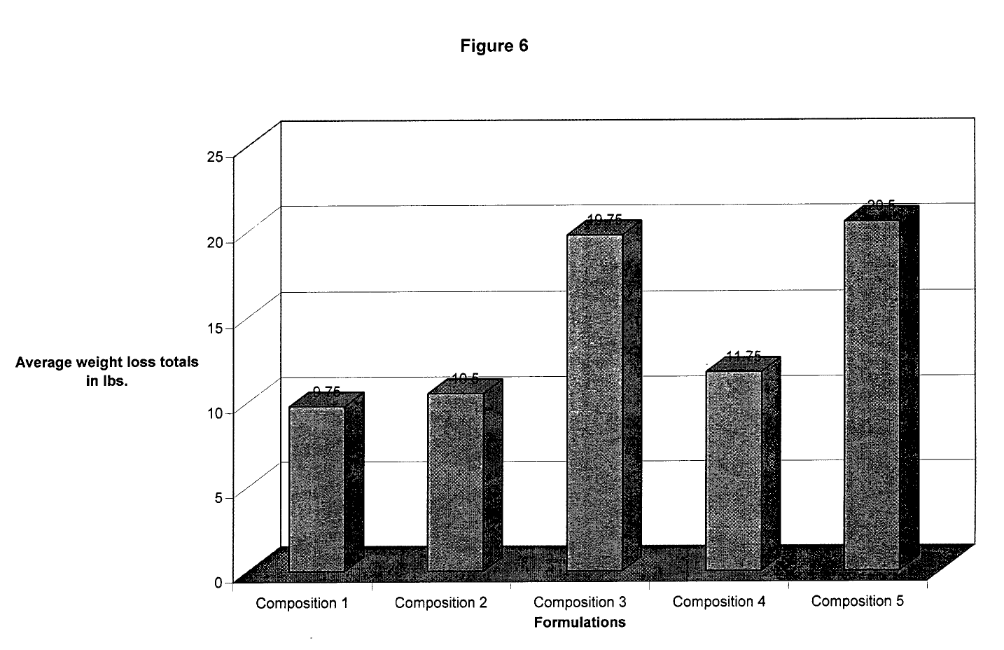

More importantly, FIG. 6 shows the average total weight loss of the five formulations. This figure clearly illustrates that total weight loss is benefited by combining a xanthine containing composition, according to the present invention, and the protein supplement with blood sugar regulator, according to the present invention. In both formulations using the combination, there was approximately a doubled weight loss experience than that experienced using any formulation that did not have the combination. Weight loss totals when the ephedra and protein where used together exceeded twenty pounds in the space of four weeks. The xanthine composition and protein exceeded nineteen pounds in four weeks. This is a very substantial weight loss in a very short period of time. This is important and exciting since it was wholly unexpected and surprising.

It is obvious in reviewing the data that this approach to weight loss is extremely novel especially as regards the anxiety commonly associated with weight loss. It could therefore be assumed that such an approach would have a more successful long term result since compliance would be so much greater. An individual that is not anxious while losing weight with a calorie deprivation approach is far more likely to continue on such a program.

Additionally, while the FEV average in 10 subjects before and after using a xanthine containing composition showed an increase in FEV (before=3.51 l; after=4.21 l; as 17% increase in FEV); the FEV average in 10 subjects before and after using a composition containing ephedrea, xanthine, and alpha adrenergic stimulants showed a greater increase (before=3.35 l; after=4.3 l; a 22% increase in FEV). The FEV measurement were performed using standard spirometry techniques as noted in Diagnosis of Diseases of the Chest by Fraser and Pare', vol. 1, p. 319-332. Measurements were done before and 1.5 hours after administration of the compositions.

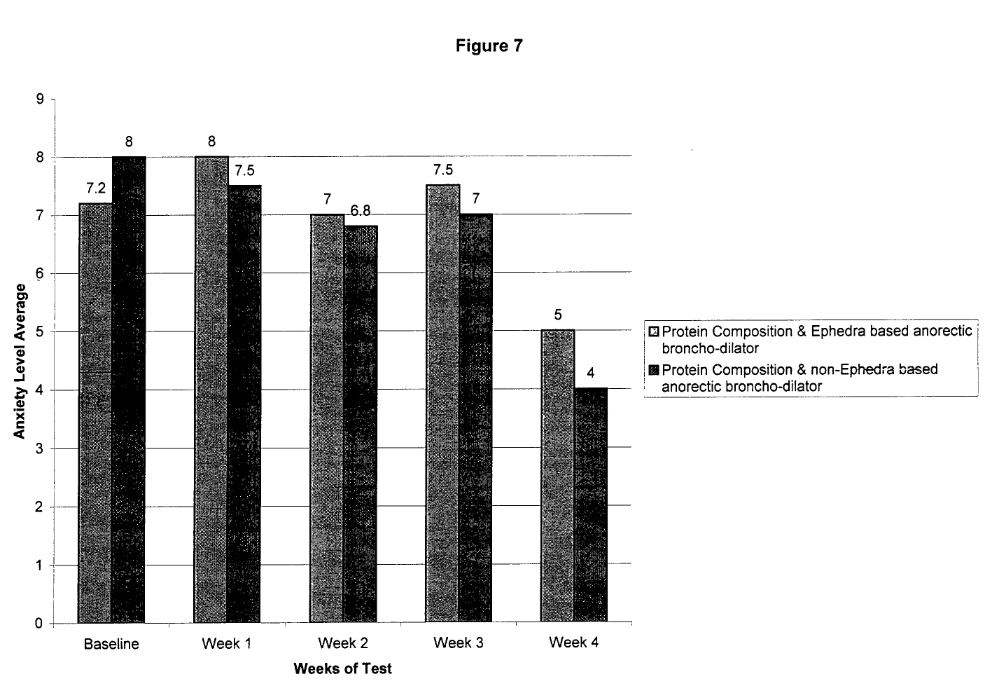

What is equally interesting, is the patients where given the Hamilton Anxiety Scale test prior to and following the four weeks test of this composition, see FIG. 7. In each case there was no difference noted statistically between the scores in patients prior to the diet as contrasted with after the diet. As previously noted, anxiety and depression result in excessive appetite. The fact that people were not anxious when on this approach indicates that it would be useful on long-term bases for those that required it. Rapid weight loss due to any condition produces a certain degree of discomfort and therefore anxiety. Generally speaking it is well known in the art that all anorectic agents that increase sympathetic zone; i.e. are broncho-dilators will inherently induce anxiety. Likewise, weight loss associated with calorie deprivation will invariably induce anxiety.

The Hamilton anxiety rating scale examination was administered at the beginning of the study and weekly thereafter for 4 weeks. The scores were averaged among the 20 individuals and the results were surprising and unexpected. To this end twenty five individuals were examined by a board certified psychiatrist and given the Hamilton Anxiety Rating Scale (see e.g. Comprehensive Textbook of Psychiatry, Kaplan, Freedman, and Sadock, ed. Williams & Wilkins, pub., Baltimore, Md.) In the Hamilton Anxiety Rating, a score of 0-10 is within normal limits, 10-20 indicates a potential need for counseling or other intervention, while a score greater than 20 indicates a potential need for pharmaceutical intervention. Five of the individuals were removed from the study because of a pre-existing psychiatric condition. The remaining 20 were divided into 2 groups of 10 each. Ten were put into the group that did the high protein blood sugar regulating composition and the ephedra based anorectic broncho-dilating agent. The other 10 were placed on the high protein blood sugar regulating composition in conjunction with an anorectic broncho-dilator that was not ephedra based.

As can be seen from FIG. 7, the performance of the high protein blood sugar regulating composition in conjunction with an anorectic broncho-dilator that was not ephedra based showed little overall reduction in anxiety until the last week of the test. The performance of the high protein blood sugar regulating composition and the ephedra based anorectic broncho-dilating agent, on the there hand, showed a demonstrable decrease in anxiety during use, with the most marked decrease during the last week of the test. This, however, was attributed to gratification due to the substantial amount of weight lost with minimal effort. At no time was there a significant increase in anxiety among any of the subjects that would require counseling or pharmaceutical intervention. In no case was an exercise program of any sort recommended to the study participants.

The active ingredients in the inventive compositions can be administered as a mixture thereof, or in combination with one or more pharmaceutically acceptable inert materials, binders, carriers or excipients, collectively referred to as adjuvants. Thus, the composition may contain binders such as microcrystalline cellulose, gum tragacanth or gelatin; excipients such as starch or lactose; carriers such as sucrose, kaolin, glycerin, starch dextrins, sodium alginate, carboxymethylcellulose and ethyl cellulose; disintegrating agents such as alginic acid, Primogel, corn starch and the like; lubricants such as magnesium stearate or Sterotex; and glidants such as colloidal silicon dioxide. When the dosage unit form is a capsule, it may contain, in addition to materials of the above type, a liquid carrier such as polyethylene glycol or a fatty oil. Other dosage unit forms may contain other various materials that modify the physical form of ht dosage unit, for example, as coatings. Thus, tablets or ills may be coated with sugar, shellac, or other enteric coating agents. Materials used in preparing these various compositions should be pharmaceutically pure and non-toxic in the amounts used.

The inventive formulation of the present invention may be administered systemically. Accordingly, the inventive formulation may be formulated for oral as well as injectable administration. In the case of oral administration, the inventive formulation of this invention may be manufactured by combining all ingredients in a form suitable for oral administration, and preferably as a pill, capsule or tablet. For example, the inventive formulation of the present invention may be encapsulated (such as in a coating of hard gelatin) for oral administration. The inventive formulation may be in the form of a wafer of chewing gum. Such techniques are well known in the art (see, e.g., Baker, Richard, Controlled Release of Biologically Active Agents, John Wiley & Sons, 1986). Inert fillers may also be present in the oral (e.g., tablet or capsule) form, in which case a powdered form may be preferred. Suitable inert fillers include magnesium stearate and silicon dioxide. The inert fillers may be present in the inventive formulation of the invention up to less than 3 percent by weight of the total composition.

Alternatively, the inventive formulation may first be combined with one or more suitable carriers or diluents to yield a pharmaceutical preparation suitable for oral or parenteral application. Such diluents or carriers, however, should not interact with the mood stabilizing composition to significantly reduce the effectiveness thereof. Suitable carriers for parenteral application (such as intravenous, subcutaneous or intramuscular injection) include sterile water, physiological saline, bacteriostatic saline (saline containing 0.9 mg/ml benzyl alcohol) and phosphate-buffered saline.

The inventive formulation may be a liquid, such as an elixer, suspension or syrup. In any case, the inventive formulation may be formulated to have a pleasant taste, or it may be coated so that it has essentially no taste. For example, sweetening agents such as sucrose or saccharin may be added or a flavoring agent such as peppermint, methyl salicylate or orange flavoring. Coloring agents, e.g., dyes may also be present.

The inventive formulation may be administered to achieve a variety of beneficial effects. Thus, the inventive formulation may serve as a stimulant, to increase cerebral cortical activity, to elevate mood, to enhance short-term memory, to provide increases in musculature relative to adipose tissue and enhance athletic performance, and decreases in appetite. These beneficial effects are discussed further below.

It has been surprisingly found that the inventive formulation provide a sustained and noticeable stimulant effect far beyond that typically observed upon ingestion of an equivalent amount of caffeine or second xanthine compound alone. The inventive formulation is therefore also directed to a method of employing the inventive formulation of the invention to enhance cerebral cortical activity and thereby provide a stimulatory effect. Thus, the invention provides a method for enhancing cerebral cortical activity in a subject in need thereof. A “subject in need thereof” may be a warm-blooded animal who has been diagnosed to have attention deficiency disease. According to the method, an effective amount of the inventive formulation as described above is administered to a subject in need of enhanced cerebral cortical activity. Furthermore, the inventive formulation affords this stimulant effect with an amelioration of the diffuse chronic depolarization and subsequent cortical depression commonly associated with stimulants alone. Accordingly, methods for enhancing cerebral cortical activity while ameliorating the diffuse chronic depolarization and subsequent cortical depression commonly associated with stimulants alone is provided by the present invention.

It has also been surprisingly found that the inventive formulation may afford substantial enhancements in short term memory as compared with caffeine alone. The invention is therefore also directed to a method of employing the inventive formulation to aid short term memory recall. Thus, the invention provides a method for enhancing the short term memory of a subject in need thereof, comprising oral administration to the subject of an effective amount of a composition of the invention as described above. This is particularly important in preserving functionality while individuals are experiencing calorie deprivation.

In a preferred embodiment, the inventive formulation contains a biosynthetic precursor to a neurosteroid. When the inventive formulation contains a biosynthetic precursor to a neurosteroid, it is particularly preferred to administer such a composition to a subject in need of increased in muscular development and athletic performance, and/or decreased in appetite.

It has also been surprisingly found that the inventive formulation causes a significant reduction in the appetite of an overweight person who consumes the composition. Overweight person who consume the inventive composition experience weight loss because their caloric consumption decreases as their interest in food is reduced. The inventive formulation is therefore useful for weight reduction and long-term weight management. Thus, the invention provides a method for achieving weight reduction comprising administering to a subject in need thereof an effective amount of the inventive formulation as described above.

In a further aspect of this invention, a method for stabilizing mood is disclosed. This method provides for the systemic administration of the compositions of the present invention in a quantity sufficient to stabilize mood in warm-blooded animals. In one embodiment, the compositions are orally administered to warm-blooded animals. The oral administration of a composition of the present invention for mood stabilization is described in more detail above.

The term “effective amount” refers to an amount that is effective, upon single or multiple dose administration to the subject, in providing one or more effects as described herein. In determining the effective amount or dose, a number of factors are considered by the attending diagnostician, including, but not limited to: the species of mammal; its size, age, and general health; the specific goal desired; the severity of the problem being experienced by the subject; the responsiveness of the individual subject or the treatment; the particular composition administered; the bioavailability characteristics of the preparation administered; the dose regimen selected; the use of concomitant medication; and other relevant circumstances.

The preferred embodiment of the invention is described above in the Figures and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A composition for weight loss comprising:
a) a first composition comprising at least one substance that enhance oxygen uptake; and
b) a second composition comprising a protein supplement comprising at least one protein source and at least one substance that regulates blood sugar.

2. The composition according to claim 1 wherein at least one substance that regulates blood sugar further comprise a long chain inulin and at least one water soluble chromium compound.

3. The composition according to claim 2 wherein the at least one water soluble chromium compound are chromium arginate and/or chromium chelidamate.

4. The composition according to claim 2 further comprising additional components comprising inulin, L-methionine, and/or a medium chain triglyceride oil.

5. The composition according to claim 4 further comprising additional components comprising inulin, L-methionine, and/or a medium chain triglyceride oil.

6. The composition according to claim 1 wherein the first separate composition further comprises at least one xanthine and/or at least one ephedra based thermogenic composition.

7. The composition according to claim 6 wherein the at least one xanthine and/or at least one ephedra based thermogenic composition further comprises caffeine and at least one other member of the xanthine family that is not caffeine.

8. The composition according to claim 7 wherein the ratio of the weight of caffeine to the total weight of the other members of the xanthine family within the composition typically ranges from about 1:3 to 3:1.

9. The composition according to claim 7 wherein the ratio of the weight of caffeine to the total weight of the other members of the xanthine family within the composition typically ranges from about 1:2 to 2:1.

10. The composition according to claim 6 further comprising at least one cognitive cofactor.

11. The composition according to claim 7 wherein the at least one cognitive cofactor comprise biosynthetic precursors to neurotransmitters or neurosteroids, cerebral vasodilators, minerals, nootropic herbs, or essential amino acids.

12. The composition according to claim 11 wherein the biosynthetic precursors to neurotransmitters or neurosteroids, cerebral vasodilators, minerals, nootropic herbs, or essential amino acids are comprised of ginkgo biloba; niacin and derivatives containing the niacin nucleus; acetyl-L-carnitine; dimethylaminoethanol (DMAE); choline including esters and salts thereof; amino acids including salts and esters thereof, such as L-phenylalanine, glutamic acid, glycine, and aspartic acid; squalane; squalene; pregnenolone; dehydroepiandrosterone (DHEA); or dehydroeplandrosterone-3-sulphate.

13. A composition for weight loss comprising:
a) a first composition comprising at least one substance that enhance oxygen uptake comprising
Ephedra E @ 8%, which ranges between 0.1-40% by weight of the composition,
Green Tea Extract @ 90% Theophylline, which ranges between 0.1-40% by weight of the composition,
Guarana Extract @ 90% Caffeine, which ranges between 0.1-40% by weight of the composition,
Coleus Forskholii extract @ 10%, which ranges between 0.001-20% by weight of the composition,
L-Tyrosine, which ranges between 0.1-50% by weight of the composition,
Dimethylaminoethanol 50 mg 0.1-75% by weight of the composition,
Choline Citrate, which ranges between 0.1-75% by weight of the composition, Huperzine-A, which ranges between 0.000001-5% by weight of the composition,
St. John's Wort @ 0.3% hypericum, which ranges between 0.1-50% by weight of the composition,
Passionflower Extract A, which ranges between 0.1-50% by weight of the composition,
Potassium Chloride, which ranges between 0.1-20% by weight of the composition,
Magnesium Phosphate Dibasic, which ranges between 0.1-80% by weight of the composition,
Chromium Arginate, which ranges between 0.001-20% by weight of the composition,
White Willow Bark (Salicylic Acid), which ranges between 0.01-75% by weight of the composition, and
Excipients as necessary; and
b) a second composition comprising a protein supplement comprising at least one protein source and at least one substance that regulates blood sugar comprising:
Soy Protein, which ranges between 10-99% by weight of the composition,
Inulin, which ranges between 0.01-20% by weight of the composition,
L-Methionine, which ranges between 0.01-20% by weight of the composition,
MCT oil, which ranges between 0-10% by weight of the composition,
Vanilla Flavoring, which ranges between 0-10% by weight of the composition,
Sucralose, which ranges between 0-10% by weight of the composition,
Carboxymethyl Cellulose, which ranges between 0-20% by weight of the composition,
Carrageenan, which ranges between 0-20% by weight of the composition,
Magesium Phosphate, which ranges between 0-30% by weight of the composition,
Chromium Arginate, which ranges between 0-5% by weight of the composition,
Chromium Chelidamate, which ranges between 0-5% by weight of the composition,
Glycine, which ranges between 0.01-20% by weight of the composition,
Vanadyl Sulfate, which ranges between 0.001-10% by weight of the composition,
Manganese gluconate, which ranges between 0.001-10% by weight of the composition.

14. A composition for weight loss comprising:
a) a first composition comprising at least one substance that enhance oxygen uptake comprising
Gingko A at approximately 5.0 mg,
Theophylline at approximately 25.0 mg,
Caffeine at approximately 27.5 mg,
Green Tea at approximately 84.0 mg,
L-pyroglutamate at approximately 75.0 mg,
Xanthinol nicotinate at approximately 38.0 mg,
N-Acetyl-L-carnitine at approximately 7.5 mg,
Choline Bitartrate at approximately 122.0 mg,
DMAE bitartrate at approximately 60.0 mg,
Magnesium glycinate at approximately 25.0 mg,
L-phenylalanine at approximately 50.0 mg, and
Chromium arginate at approximately 200 mg, and
b) a second composition comprising a protein supplement comprising at least one protein source and at least one substance that regulates blood sugar comprising:
Soy Protein, which ranges between 10-99% by weight of the composition,
Inulin, which ranges between 0.01-20% by weight of the composition,
L-Methionine, which ranges between 0.01-20% by weight of the composition,
MCT oil, which ranges between 0-10% by weight of the composition,
Vanilla Flavoring, which ranges between 0-10% by weight of the composition,
Sucralose, which ranges between 0-10% by weight of the composition,
Carboxymethyl Cellulose, which ranges between 0-20% by weight of the composition,
Carrageenan, which ranges between 0-20% by weight of the composition,
Magesium Phosphate, which ranges between 0-30% by weight of the composition,
Chromium Arginate, which ranges between 0-5% by weight of the composition,
Chromium Chelidamate, which ranges between 0-5% by weight of the composition,
Glycine, which ranges between 0.01-20% by weight of the composition,
Vanadyl Sulfate, which ranges between 0.001-10% by weight of the composition,
Manganese gluconate, which ranges between 0.001-10% by weight of the composition.

15. A composition for weight loss comprising:
a) a first composition comprising at least one substance that enhance oxygen uptake comprising
Caffeine, which ranges between 1-60% by weight of the composition,
Theophylline, which ranges between 1-60% by weight of the composition,
Gingko-A, which ranges between 0-50% by weight of the composition,
L-pyroglutamate, which ranges between 1-70% by weight of the composition,
Xanthinol nicotinate, which ranges between 1-70% by weight of the composition,
N-Acetyl-L-carnitine, which ranges between 0-50% by weight of the composition,
Choline Bitartrate, which ranges between 1-70% by weight of the composition,
DMAE, which ranges between 1-70% by weight of the composition,
Magnesium glycinate, which ranges between 0-50% by weight of the composition,
Potassium aspartate 21%, which ranges between 0-50% by weight of the composition,
Chromium arginate, which ranges between 0-5% by weight of the composition, and
L-phenylalanine, which ranges between 1-70% by weight of the composition, and
b) a second composition comprising a protein supplement comprising at least one protein source and at least one substance that regulates blood sugar comprising:
Soy Protein, which ranges between 10-99% by weight of the composition,
Inulin, which ranges between 0.01-20% by weight of the composition,
L-Methionine, which ranges between 0.01-20% by weight of the composition,
MCT oil, which ranges between 0-10% by weight of the composition,
Vanilla Flavoring, which ranges between 0-10% by weight of the composition,
Sucralose, which ranges between 0-10% by weight of the composition,
Carboxymethyl Cellulose, which ranges between 0-20% by weight of the composition,
Carrageenan, which ranges between 0-20% by weight of the composition,
Magesium Phosphate, which ranges between 0-30% by weight of the composition,
Chromium Arginate, which ranges between 0-5% by weight of the composition,
Chromium Chelidamate, which ranges between 0-5% by weight of the composition,
Glycine, which ranges between 0.01-20% by weight of the composition,
Vanadyl Sulfate, which ranges between 0.001-10% by weight of the composition,
Manganese gluconate, which ranges between 0.001-10% by weight of the composition.
